(19) United States
(12) Patent Application Publication
(10) Pub. No.: US 2024/0127956 A1
(43) Pub. Date: Apr. 18, 2024

(54) METHOD AND SYSTEM FOR PROVIDING INFORMATION ABOUT VIRUS INFECTION AND/OR VIRUS INFECTION DISEASES

(71) Applicants: Osaka University, Osaka (JP); National Institutes of Biomedical Innovation, Health and Nutrition, Osaka (JP); Kagami Inc., Osaka (JP)

(72) Inventors: Yoshitaka ISAKA, Osaka (JP); Tomonori KIMURA, Osaka (JP); Shihoko KIMURA, Osaka (JP); Masashi MITA, Osaka (JP)

(73) Assignees: Osaka University, Osaka (JP); National Institutes of Biomedical Innovation, Health and Nutrition, Osaka (JP); Kagami Inc., Osaka (JP)

(21) Appl. No.: 18/488,373

(22) Filed: Oct. 17, 2023

(30) Foreign Application Priority Data
Oct. 18, 2022 (JP) 2022-167238

(51) Int. Cl. G16H 50/20; G01N 33/68; G16H 20/10

(52) U.S. Cl. CPC G16H 50/20; G01N 33/6893; G16H 20/10; G01N 2800/52

(57) ABSTRACT

Provided herein is a method for providing information about virus infection and/or virus infection disease in a subject, comprising: making a determination on the detection and/or stage classification of virus infection and/or virus infection disease in the subject using an indicator associated with a D-amino acid in the subject; and providing information about virus infection and/or virus infection disease in the subject based on the results of the determination.

### FIELD

The present invention relates to a method and a system for providing information about virus infection and/or virus infection disease.

### BACKGROUND

Infection is defined as the establishment, invasion, and multiplication of a pathogen (pathogenic microorganism) such as a virus in cells, tissues, and organs of a host through various infection routes. Pathogens that invade the host are eliminated from the host by the host's defense mechanism in most cases, but when infection is established and leads to occurrence of some symptoms or signs such as fever (onset of disease), the condition is referred to as infection disease. Infections caused by viruses such as coronaviruses and influenza viruses and the resulting infection diseases can pose a threat to healthcare and the economy through pandemics. Genetic tests (e.g., PCR method), immunological tests (e.g., antigen detection method), antibody/antigen tests, etc. have been put into practice for the detection of viral infections and viral diseases. Urinary liver-type fatty acid-binding protein (L-FABP) has been proposed as a biomarker for predicting the risk of increased severity of viral infections (Patent Literature 1).

In recent years, advances in the performance of techniques for identifying and analyzing chiral amino acids have led to the development of quantitative studies that identify trace amounts of D- and L-amino acids in mammals and other living organisms. This has clarified the existence and functions of some D-amino acids, which have conventionally been treated as total amino acids (D-amino acids+L-amino acids) or L-amino acids for convenience due to technical limitations. In the mammalian intestine, host intestinal immunity has been shown to be regulated via D-amino acid metabolism by D-amino acid oxidase (DAO) against Vibrio spp. (Non-Patent Literature 1). It has also been reported that the % D values ({(D-amino acid)/(D-amino acid+L-amino acid)}×100) of D-asparagine, D-serine, D-alanine, and D-proline in blood of humans infected with human immunodeficiency virus (HIV) and receiving antiretroviral therapy correlated with the age and renal function markers (eGFR) of subjects, but that no variation was observed with HIV infection (Non-patent Literature 2).

### CITATION LIST

### Patent Literature

[Patent Literature 1] JP 6933834 B
[Patent Literature 2] JP 6868878 B
[Patent Literature 3] JP 6993654 B
[Patent Literature 4] WO 2020/196436 A
[Patent Literature 5] WO 2013/140785 A

### Non-Patent Literature

[Non-Patent Literature 1] Sasabe J, Miyoshi Y, Rakoff-Nahoum S, Zhang T, Mita M, Davis B M, Hamase K, Waldor M K. Interplay between microbial D-amino acids and host D-amino acid oxidase modifies murine mucosal defence and gut microbiota. Nat Microbiol. 2016 Jul. 25; 1(10):16125. doi: 10.1038/nmicrobiol.2016.125.
[Non-Patent Literature 2] Yap S H, Lee C S, Furusho A, Ishii C, Shaharudin S, Zulhaimi N S, Kamarulzaman A, Kamaruzzaman S B, Mita M, Leong K H, Hamase K, Rajasuriar R. Plasma D-amino acids are associated with markers of immune activation and organ dysfunction in people with HIV. AIDS. 2022 Jun. 1; 36(7):911-921. doi: 10.1097/QAD.0000000000003207.
[Non-Patent Literature 3] Kawamura M, Hesaka A, Taniguchi A, Nakazawa S, Abe T, Hirata M, Sakate R, Horio M, Takahara S, Nonomura N, Isaka Y, Imamura R, Kimura T. Measurement of glomerular filtration rate using endogenous D-serine clearance in living kidney transplant donors and recipients. EClinicalMedicine. 2021 Dec. 5; 43:101223. doi: 10.1016/j.eclinm.2021.101223.
[Non-Patent Literature 4] Hesaka A, Yasuda K, Sakai S, Yonishi H, Namba-Hamano T, Takahashi A, Mizui M, Hamase K, Matsui R, Mita M, Horio M, Isaka Y, Kimura T. Dynamics of D-serine reflected the recovery course of a patient with rapidly progressive glomerulonephritis. CEN Case Rep. 2019 November; 8(4):297-300. doi: 10.1007/s13730-019-00411-6.
[Non-Patent Literature 5] Sasabe J, Miyoshi Y, Suzuki M, Mita M, Konno R, Matsuoka M, Hamase K, Aiso S. D-amino acid oxidase controls motoneuron degeneration through D-serine. Proc Natl Acad Sci USA. 2012 Jan. 10; 109(2):627-32. doi: 10.1073/pnas.1114639109.
[Non-Patent Literature 6] Fang Y, Zhang H, Xie J, Lin M, Ying L, Pang P, Ji W. Sensitivity of Chest CT for COVID-19: Comparison to RT-PCR. Radiology. 2020 August; 296(2):E115-E117. doi: 10.1148/radiol.2020200432.
[Non-Patent Literature 7] Ai T, Yang Z, Hou H, Zhan C, Chen C, Lv W, Tao Q, Sun Z, Xia L. Correlation of Chest CT and RT-PCR Testing for Coronavirus Disease 2019 (COVID-19) in China: A Report of 1014 Cases. Radiology. 2020 August; 296(2):E32-E40. doi: 10.1148/radiol.2020200642.
[Non-Patent Literature 8] Hesaka A, Sakai S, Hamase K, Ikeda T, Matsui R, Mita M, Horio M, Isaka Y, Kimura T. D-Serine reflects kidney function and diseases. Sci Rep. 2019 Mar. 25; 9(1):5104. doi: 10.1038/s41598-019-41608-0.
[Non-Patent Literature 9] Kimura T, Hamase K, Miyoshi Y, Yamamoto R, Yasuda K, Mita M, Rakugi H, Hayashi T, Isaka Y. Chiral amino acid metabolomics for novel biomarker screening in the prognosis of chronic kidney disease. Sci Rep. 2016 May 18; 6:26137. doi: 10.1038/srep26137.

### SUMMARY

Virus infections and the resulting virus infection diseases have the potential to cause medical, economic, and security social problems, as seen in pandemics and bioterrorism, and methods are needed to provide accurate clinical information needed to deal with them.

The present inventors have comprehensively and precisely quantified and analyzed chiral amino acids (amino acids identified as D-amino acids and L-amino acids) in the blood of virus-infected subjects, and have found that a relationship exists between the presence or absence of viral infection and the pathogenesis, progression, and stage of viral infection and the amounts of chiral amino acids in blood, with a constant pattern of variations in the amounts of D-amino acids in blood. Furthermore, as a result of their diligent research on this relationship, the present inventors have discovered the clinical usefulness in clinical testing, diagnosis, and treatment of infection diseases by developing an indicator of D-amino acids in blood as an indicator that provides information on virus infection and/or virus infection disease, and have thus completed the present invention that provides a solution to the above problem.

Embodiments of the present invention relate to, e.g., the following aspects.

### [Aspect 1]

A method for providing information about virus infection and/or virus infection disease in a subject, comprising:
making a determination on the detection and/or stage classification of virus infection and/or virus infection disease in the subject using an indicator associated with a D-amino acid in the subject; and
providing information about virus infection and/or virus infection disease in the subject based on the results of the determination.

### [Aspect 2]

The method according to Aspect 1, wherein the indicator associated with a D-amino acid is a measurement value for the D-amino acid in blood or its correction value or correction formula.

### [Aspect 3]

The method according to Aspect 2, wherein the indicator associated with a D-amino acid is a value or formula obtained by correcting the amount of the D-amino acid by a parameter associated with a biological substance (e.g., an L-amino acid) in the subject.

### [Aspect 4]

The method according to Aspect 2, wherein the indicator associated with a D-amino acid is a value or formula obtained by correcting the amount of the D-amino acid by a parameter associated with a kidney function in the subject.

### [Aspect 5]

The method according to any one of Aspects 1 to 4, wherein the D-amino acid is one or more D-amino acids selected from the group consisting of D-proline, D-serine, D-alanine, and D-asparagine.

### [Aspect 6]

The method according to any one of Aspects 1 to 5, wherein the virus is a virus belonging to a family selected from Orthomyxoviridae, Coronaviridae, Paramyxoviridae, Rhabdoviridae, Arenaviridae, Bunyavirales, Filoviridae, Retroviridae, Togaviridae, Flaviviridae, Picornaviridae, Astroviridae, Caliciviridae, Reoviridae, Parvoviridae, Adenoviridae, Papillomaviridae, Polyomaviridae, Herpesviridae, Hepadnaviridae, and Poxviridae.

### [Aspect 7]

The method according to any one of Aspects 1 to 6, wherein the determination on the detection of the virus infection and/or virus infection disease includes:
when the indicator associated with a D-amino acid in the subject has exhibited a decreasing trend, determining that the subject has become infected with virus and/or affected with virus infection disease.

### [Aspect 8]

The method according to any one of Aspects 1 to 7, wherein the determination on the stage classification of the virus infection and/or virus infection disease is selected from:
the indicator associated with a D-amino acid in the subject has decreased, determining that the subject is in an aggravating state of viral infection,
the indicator associated with a D-amino acid in the subject has increased, determining that the subject is in an improving state of viral infection and/or
when the indicator associated with a D-amino acid in the subject has repeatedly decreased and increased and then converged to within a healthy reference range, determining that the subject is in a healing state of viral infection.

### [Aspect 9]

The method according to any one of Aspects 1 to 8, wherein the determination on the detection and/or stage classification of the virus infection and/or virus infection disease is carried out by comparing the indicator associated with a D-amino acid in the subject with a determination criterion for virus infection and/or virus infection disease.

### [Aspect 10]

The method according to any one of Aspects 1 to 9, wherein the information about virus infection and/or virus infection disease in a subject is information on an event selected from the group consisting of:
whether or not virus infection and/or virus infection disease in the subject is detected;
stage classification of virus infection disease in the subject;
validation of test results and/or diagnosis results on virus infection and/or virus infection disease in the subject; and
selection of therapeutic means for virus infection disease in the subject.

### [Aspect 11]

The method according to Aspect 10, wherein the therapeutic means for virus infection disease include means selected from antivirus drug, blood purification therapy, mechanical ventilation, and extracorporeal membrane oxygenation (ECMO).

### [Aspect 12]

A system for carrying out the method according to any one of Aspects 1 to 11, comprising:
an input unit for inputting information from a subject;
an analytical measurement unit for analyzing and/or measuring the information from the subject inputted via the input unit to obtain an indicator associated with a D-amino acid in the subject;
a memory unit for storing a determination criterion for virus infection and/or virus infection disease;
a data processing unit for processing the indicator obtained by the analytical measurement unit for the subject based on the determination criterion stored by the memory unit to make a determination on virus infection and/or virus infection disease in the subject; and
an output unit for outputting the results of determination by the data processing unit as information about virus infection and/or virus infection disease in the subject.

The method and system of the present invention makes it possible to provide information about virus infection and/or virus infection disease in a subject with high accuracy and/or efficiently by making a determination using an indicator associated with a D-amino acid in the subject to make a determination.

### BRIEF DESCRIPTION OF THE DRAWINGS

FIG. 1 indicates graphs showing the changes over time in the amounts of D-amino acids in the blood of mice infected with influenza virus in Example 1. A:D-Ala, B:D-Asn, C:D-Pro, D:D-Ser.

FIG. 2 indicates graphs showing the amounts of D-amino acids in the blood of mice infected with influenza virus in comparison with non-infected mice in Example 1. A:D-Ala, B:D-Asn, C:D-Pro, D:D-Ser.

FIG. 3 indicates graphs showing the ratios of D-amino acids (% D) in the blood of mice infected with influenza virus in comparison with non-infected mice in Example 1. A:D-Ala, B:D-Asn, C:D-Pro, D:D-Ser.

FIG. 4 indicates graphs showing the body weights of mice infected with influenza virus in comparison with non-infected mice in Example 1.

FIG. 5-1 indicates graphs showing the changes over time in the amounts of D-amino acids in the blood of humans infected with coronavirus in Example 2. A:D-Ala, B:D-Asn.

FIG. 5-2 indicates graphs showing the changes over time in the amounts of D-amino acids in the blood of humans infected with coronavirus in Example 2. C:D-Pro, D:D-Ser.

FIG. 6 indicates graphs showing the amounts of D-amino acids in the blood of humans infected with coronavirus in comparison with non-infected humans in Example 2. A:D-Ala, B:D-Asn, C:D-Pro, D:D-Ser.

FIG. 7 indicates graphs showing the ratios of D-amino acids (% D) in the blood of humans infected with coronavirus in comparison with non-infected humans in Example 2. A:D-Ala, B:D-Asn, C:D-Pro, D:D-Ser.

FIG. 8 indicates a graph showing the changes over time in the amounts of D-amino acids in the blood of end-stage kidney failure patients among humans infected with coronavirus in Example 2.

FIG. 9 indicates diagnostic ability ROC (receiver operating characteristic) curves based on univariate analysis of the amounts of D-amino acids in the blood of humans infected with coronavirus in Example 2. A:D-Ala, B:D-Pro, C:D-Ser.

FIG. 10 indicates diagnosis ability ROC curves based on univariate analysis of the ratios of D-amino acids (% D) in the blood of humans infected with coronavirus in Example 2. A:D-Ala, B:D-Pro, C:D-Ser.

FIG. 11 indicates a table showing the areas under the curve (AUC) for the diagnostic ability ROC curves based on bivariate analysis of the amounts of D-amino acids in the blood of humans infected with coronavirus and their 95% confidence intervals in Example 2

FIG. 12 is a block diagram schematically showing an exemplary configuration of the system of the present invention.

FIG. 13 is a schematic flowchart showing an exemplary processing carried out by the system of the present invention (the method of the present invention).

### DETAILED DESCRIPTION

The present invention is described hereinafter in detail with reference to specific embodiments thereof. However, the present invention is not limited to the following embodiments and can be carried out in any embodiment that does not deviate from the gist according to the present invention.

All patent publications, patent application publications, and non-patent documents cited in this disclosure are incorporated herein by reference in their entirety for all purposes.

Amino acids and residues thereof may herein be represented by three-letter abbreviations well known to a person skilled in the art. The three-letter abbreviations of major amino acids are shown in the following table.

TABLE 1

| Abbreviation | Definition |
|---|---|
| Ala | Alanine |
| Arg | Arginine |
| Asn | Asparagine |
| Asp | Aspartic acid |
| Cys | Cysteine |
| Gln | Glutamine |
| Glu | Glutamic acid |
| Gly | Glycine |
| His | Histidine |
| Ile | Isoleucine |
| Leu | Leucine |
| Lys | Lysine |
| Met | Methionine |
| Phe | Phenylalanine |
| Pro | Proline |
| Ser | Serine |
| Thr | Threonine |
| Trp | Tryptophan |
| Tyr | Tyrosine |
| Val | Valine |
| D-AA | D-amino acid |
| L-AA | L-amino acid |

### Method for Providing Information about Virus Infection and/or Virus Infection Disease (the Method of the Present Invention)

An embodiment of the present invention relates to a method for providing information about virus infection and/or virus infection disease in a subject, comprising: making a determination on the detection and/or stage classification of virus infection and/or virus infection disease in the subject using an indicator associated with a D-amino acid in the subject; and providing the information about virus infection and/or virus infection disease in the subject based on the results of the determination (hereinafter also referred to as “the method of the present invention”).

The method of the present invention is a novel evaluation approach to virus infection and/or virus infection disease, using an indicator associated with a D-amino acid (e.g., the amounts of D-amino acids in the blood) to thereby provide information about virus infection and/or virus infection disease in the subject. This makes it possible, e.g., to improve the accuracy of detection and/or stage classification of the virus infection and/or virus infection disease of the subject. The present invention also relates to, e.g., a method for assisting the selection of appropriate therapeutic measures.

D-Amino Acids and L-Amino Acids

The terms “D-amino acids” (abbreviated herein as “D-isomer”) and “L-amino acids” (as “L-isomers”) refer to stereoisomers of amino acids based on the D/L notation of IUPAC nomenclature. The D-isomer and the L-isomer are enantiomers to each other. It is known that the majority of protein-constituent amino acids existing in vivo are L-isomers. Although glycine does not have D- and L-isomers, glycine is treated herein as D-isomer for convenience unless otherwise specified.

Specific examples of D-amino acids herein include, although are not limited to, glycine, D-alanine, D-histidine, D-isoleucine, D-allo-isoleucine, D-leucine, D-lysine, D-methionine, D-phenylalanine, D-threonine, D-allo-threonine, D-tryptophan, D-valine, D-arginine, D-cysteine, D-glutamine, D-proline, D-tyrosine, D-aspartic acid, D-asparagine, D-glutamic acid, and D-serine. Preferred among them are D-proline, D-serine, D-alanine, and D-asparagine. These D-amino acids may be used either singly or in combination of any two or more at any ratios.

D-cysteine in a biological sample is oxidized to D-cystine ex vivo. Therefore, an indicator (e.g., amount) associated with D-cysteine contained in a biological sample can be calculated by measuring an indicator (e.g., amount) associated with D-cystine instead of D-cysteine.

Indicator Associated with D-Amino Acids

The term “indicator associated with a D-amino acid” herein refers to an indicator that is obtained from a living organism and has some relationship to a D-amino acid (in this connection, a measurement or test value of an indicator associated with a D-amino acid obtained from the subject may also be abbreviated simply as, e.g., a “test value of D-amino acid”). Examples of indicators associated with D-amino acids include measurement values for D-amino acids in blood or their correction values or correction formulae.

An example of a measurement value for an indicator associated with a D-amino acid in blood is the amount of the D-amino acid in blood. The term “the amount of a D-amino acid in a blood” herein refers to the amount of the D-amino acid contained in the specific blood. The amounts of D-amino acids in blood may be expressed as concentrations. The amounts of D-amino acids in blood may be measured as the amounts in a sample of collected blood that has undergone centrifugation, sedimentation separation, or pre-treatments for analysis. Accordingly, the amounts of D-amino acids in blood can be measured as the amounts of D-amino acids in a blood sample derived from blood, such as collected whole blood, serum, or plasma. For example, in the case of analysis using HPLC, the amount of a D-amino acid contained in a given volume of blood is represented by a chromatogram, and can be quantified by comparison with a standard or analysis by calibration with respect to peak height, area, and shape.

The correction value or formula of a measurement value associated with a D-amino acid in blood can be any value or formula obtained via correction processing of the amount of the D-amino acid in blood. Specific examples include corrected D/L ratios of the amount of the D-amino acid in blood, % D ({(D-amino acid)/(D-amino acid+L-amino acid)}×100), which represents the ratio of the D-amino acid in blood, D-amino acid clearance, D-amino acid excretion rate (Non-Patent Literatures 3 and 4), formulas and values obtained by using the amount of the D-amino acid as explanatory variables and correcting it according to the purpose, and values obtained via predetermined formulae, etc.

Another example of a correction value for the amount of a D-amino acid in blood is a value obtained by correcting the amount of the D-amino acid in blood for physiological variables, such as age, gender, BMI, and other factors. When the dynamics of a D-amino acid is affected by kidney functions, the amount of the D-amino acid in blood may be corrected with an indicator for kidney functions and the thus-corrected value may be used. Examples of such indicators for kidney functions include, although are not intended to be limited to, one or more selected from creatinine, cystatin C, inulin clearance, creatinine clearance, urinary protein, urinary albumin, β2-MG, α1-MG, NAG, L-FABP, NGAL, glomerular filtration rate, estimated glomerular filtration rate (eGFR), renal function measurement values and estimated formulae using D-amino acids (Patent Literatures 2, 3, and 4). Specific examples include the correction to determine the ratio of the blood D-amino acid level to the blood creatinine level {(the amount of the D-amino acid in the blood)/(the amount of creatinine in the blood)}. Furthermore, it is known that in vivo D-amino acid levels may shift in neurodegenerative diseases (e.g., ALS), autoimmune diseases (e.g., multiple sclerosis), and metabolic diseases (e.g., diabetes) (Patent Literature 5 and Non-Patent Literature 5). Therefore, it is possible to correct the amount of the D-amino acid in blood with a shift factor or a marker for each disease.

Another example of an indicator associated with a D-amino acid in a subject is a value or formula obtained by correcting the amount of the D-amino acid in the blood of the subject with an indicator associated with a biological substance from the subject (e.g., L-amino acid). Examples of indicators associated with a biological substance for correction include, although are not limited to, the L-amino acid level and the total amino acid level in the subject's blood.

When a value obtained by correcting the amount of a D-amino acid in blood with another test value is used as an indicator associated with the D-amino acid, the criteria for detection and/or stage classification of virus infection and/or virus infection disease based on a change (e.g., a decrease or an increase) in the amount of the D-amino acid in blood and the judgment and analysis methods can be set and changed according to the correction details for the amount of the D-amino acid in blood. For example, when a value obtained by correcting the amount of a D-amino acid in blood as an inverse (e.g., [1/(the amount of the D-amino acid in blood)]) or a multiplier is used as an indicator associated with the D-amino acid, the judgment criteria set based on a change (e.g., a decrease or an increase) in the amount of the D-amino acid in blood may be set as an inverse (e.g., a decrease in the amount of the D-amino acid in blood indicates an increase in the original value and an increase in the amount of the D-amino acid in blood indicates a decrease in the original value) or a logarithm.

In the method of the present invention, the indicators associated with D-amino acids (e.g., the amount of the D-amino acid in blood, corrected D/L ratio, % D ({(D-amino acid)/(D-amino acid+L-amino acid)}×100), D-amino acid clearance, D-amino acid excretion rate, etc.) may be used either singly or in combination of any two or more. In the latter case, it is possible to subject a sample to a panel testing combining two or more indicators associated with D-amino acids at the same time.

In the method of the present invention, the sample for measuring the indicator associated with a D-amino acid may be one or more samples obtained via a single test or two or more samples obtained in two or more tests. When two or more samples are used, the samples may be obtained either at the same point in time or at two or more different points in time. The types of these samples may be selected as appropriate depending on various embodiments described below.

The amounts of D-amino acids and/or L-amino acids in a sample such as blood may be determined by any method. For example, they can be quantified by chiral column chromatography, enzymatic methods, or immunological methods using monoclonal antibodies that identify optical isomers of amino acids. The amounts of D-amino acids and/or L-amino acids in a sample may be measured using any method known to those skilled in the art. Examples include: chromatography methods and enzymatic methods (Y. Nagata et al., Clinical Science, 73 (1987), 105. Analytical Biochemistry, 150 (1985), 238., A. D'Aniello et al., Comparative Biochemistry and Physiology Part B, 66 (1980), 319. Journal of Neurochemistry, 29 (1977), 1053., A. Berneman et al., Journal of Microbial & Biochemical Technology, 2 (2010), 139., W. G. Gutheil et al., Analytical Biochemistry, 287 (2000), 196., G. Molla et al., Methods in Molecular Biology, 794 (2012), 273., T. Ito et al., Analytical Biochemistry, 371 (2007), 167, etc.); antibody methods (T. Ohgusu et al., Analytical Biochemistry, 357 (2006), 15, etc.), gas chromatography (GC) (H. Hasegawa et al., Journal of Mass Spectrometry, 46 (2011), 502., M. C. Waldhier et al., Analytical and Bioanalytical Chemistry, 394 (2009), 695., A. Hashimoto, T. Nishikawa et al., FEBS Letters, 296 (1992), 33., H. Bruckner and A. Schieber, Biomedical Chromatography, 15 (2001), 166., M. Junge et al., Chirality, 19 (2007), 228., M. C. Waldhier et al., Journal of Chromatography A, 1218 (2011), 4537, etc.), capillary electrophoresis (CE) (H. Miao et al., Analytical Chemistry, 77 (2005), 7190., D. L. Kirschner et al., Analytical Chemistry, 79 (2007), 736., F. Kitagawa, K. Otsuka, Journal of Chromatography B, 879 (2011), 3078., G. Thorsen and J. Bergquist, Journal of Chromatography B, 745 (2000), 389, etc.), and high performance liquid chromatography (HPLC) (N. Nimura and T. Kinoshita, Journal of Chromatography, 352 (1986), 169., A. Hashimoto et al., Journal of Chromatography, 582 (1992), 41., H. Bruckner et al., Journal of Chromatography A, 666 (1994), 259 N. Nimura et al., Analytical Biochemistry, 315 (2003), 262., C. Muller et al., Journal of Chromatography A, 1324 (2014), 109., S. Einarsson et al., Analytical Chemistry, 59 (1987), 1191., E. Okuma and H. Abe, Journal of Chromatography B, 660 (1994), 243., Y. Gogami et al., Journal of Chromatography B, 879 (2011), 3259., Y. Nagata et al., Journal of Chromatography, 575 (1992), 147., S. A. Fuchs et al., Clinical Chemistry, 54 (2008), 1443., D. Gordes et al., Amino Acids, 40 (2011), 553., D. Jin et al., Analytical Biochemistry, 269 (1999), 124., J. Z. Min et al., Journal of Chromatography B, 879 (2011), 3220., T. Sakamoto et al., Analytical and Bioanalytical Chemistry, 408 (2016), 517., W. F. Visser et al., Journal of Chromatography A, 1218 (2011), 7130., Y. Xing et al., Analytical and Bioanalytical Chemistry, 408 (2016), 141., K. Imai et al., Biomedical Chromatography, 9 (1995), 106., T. Fukushima et al., Biomedical Chromatography, 9 (1995), 10., R. J. Reischl et al., Journal of Chromatography A, 1218 (2011), 8379., R. J. Reischl and W. Lindner, Journal of Chromatography A, 1269 (2012), 262., S. Karakawa et al., Journal of Pharmaceutical and Biomedical Analysis, 115 (2015), 123., Hamase K, et al., Chromatography 39 (2018) 147-152, etc.).

The separation analysis system for optical isomers according to the present invention may be based on a combination of two or more separation analyses. As a specific example, the amounts of D-amino acids and/or L-amino acids in a sample can be measured by using a method for analyzing optical isomers characterized by including the steps of: flowing a sample containing components having optical isomers through a first column packing material as a stationary phase together with a first liquid as a mobile phase to separate the components in the sample; holding each of the components from the sample individually in a multi-loop unit; flowing each of the components from the sample individually retained in the multi-loop unit, together with a second liquid as a mobile phase, through a channel to a second column packing material having an optically active center as a stationary phase, to partition the optical isomers contained in each of the components from the sample; and detecting the optical isomers contained in each of the components from the sample (JP 4291628 B). For HPLC analysis, D- and L-amino acids may be derivatized beforehand with fluorescent reagents such as o-phthalaldehyde (OPA) and 4-fluoro-7-nitro-2,1,3-benzoxadiazole (NBD-F), or made into diastereomers with, e.g., N-tert-butyloxycarbonyl-L-cysteine (Boc-L-Cys) (HAMASE, Kenji and ZAIZU, Kiyoshi, Analysis Chemistry, Vol. 53, pp. 677-690 (2004)). Alternatively, the amounts of D-amino acids and/or L-amino acids in a sample can be measured by immunological methods using monoclonal antibodies that identify optical isomers of amino acids, such as monoclonal antibodies that bind specifically to D-amino acids or L-amino acids. When the total amount of the D-isomer and L-isomer of an amino acid is used as an indicator, it is not necessary to analyze the D- and L-isomers of the amino acids separately, but the amino acid can be analyzed without distinguishing the D- and L-isomers. Also in such cases, it can still be separated and quantified by enzymatic or antibody methods, GC, CE, or HPLC.

The amounts of biomolecules and drugs such as D-amino acids, L-amino acids, creatinine, and proteins may be expressed herein not only in terms of mere mass, weight, and amount (mol) of the substance, but also in terms of mass, weight, and amount (mol) of the substance per tissue, cell, organ, or molecular unit or per volume or weight, or in terms of mass, weight, amount (mol), concentration, specific gravity, and density of the substance in liquid such as blood or urine, or any other physical quantity that can be measured.

Viruses and Virus Infection Diseases

The term “virus” as used herein refers to a microscopic infectious structure whose minimal components are a nucleic acid carrying genetic information and a protein shell covering the nucleic acid, and which uses the cells of other organisms to replicate itself. The viruses to which the present invention is applicable are not limited and may be any viruses as long as they infect humans, examples thereof including viruses that causes an infection disease to the host organism upon infection.

Examples of viruses include, although are not limited to, viruses belonging to the families Orthomyxoviridae (e.g., influenza A virus, influenza B virus, and influenza C virus), Coronaviridae (e.g., SARS coronavirus (SARS-CoV), MERS coronavirus (MERS-CoV), and SARS coronavirus 2 (SARS-CoV-2)), Paramyxoviridae (e.g., measles virus and mumps virus), Rhabdoviridae, Arenaviridae, Bunyavirales, Filoviridae, Retroviridae (e.g., human immunodeficiency virus (HIV)), Togaviridae, Flaviviridae (e.g., hepatitis C virus (HCV)), Picornaviridae (e.g., rhinovirus A, B, and C), Astroviridae, Caliciviridae, Reoviridae, Parvoviridae, Adenoviridae (e.g., human adenovirus A to G), Papillomaviridae, Polyomaviridae, Herpesviridae (e.g., herpes simplex virus 1 (HSV-1), herpes simplex virus 2 (HSV-2), and varicella-zoster virus (VZV)), Hepadnaviridae (e.g., hepatitis B virus (HBV)), and Poxviridae (e.g., variola virus, vaccinia virus, monkeypox virus, and camelpox virus).

The term “infection” as used herein refers to a state in which pathogens such as viruses adsorb, settle, invade, or multiply in host cells, tissues, or organs through various infection routes, and includes apparent infection, in apparent infection, and persistent infection (latent infection). The term “infection disease” as used herein refers to a condition in which infection is established and some symptoms or signs appear (e.g., fever, chills, headache, myalgia, joint pain, etc., in the case of influenza, and pneumonia, etc. in the case of COVID-19).

Examples of virus infection diseases include, although are not limited to, common cold syndrome, influenza, severe acute respiratory syndrome (SARS), Middle East respiratory syndrome (MERS), novel coronavirus infection disease (COVID-19), epidemic parotitis (mumps), other viral pneumonia, chicken pox, shingles, measles, oral herpes, genital herpes, hepatitis B, hepatitis C, smallpox, and monkeypox.

Subjects

The term “subject” herein includes, although are not limited to, vertebrates. Examples of vertebrate include mammals, birds, reptiles, amphibians, and fish. Examples of mammals include humans and non-human mammals such as mice, rats, guinea pigs, monkeys, rabbits, cows, horses, pigs, sheep, goats, camels, dogs, and cats. Examples of birds includes chickens. Among them, the subject may preferably be a human or a non-human mammal, especially a human. In addition, various animals that have had virus infection and/or virus infection disease induced by means of transplantation of virus infection and/or virus infection disease cells, genetic modification, or administration of drugs (DNA, RNA, various vaccines, etc.) may be used as targets. Furthermore, various animal individuals, cells, tissues, organoids, etc. that serve as virus infection disease models may be used as subjects.

Determination Criteria

According to an embodiment of the present invention, the method of the present invention may include making a determination on the detection of virus infection and/or virus infection disease in a subject by comparing an indicator associated with the D-amino acid in the subject with a determination criterion. The term “determination criterion” as used herein refers to a criterion for making a determination on the detection of virus infection and/or virus infection disease in a subject using an indicator associated with a D-amino acid in the subject. Examples include, but are not limited to, a predetermined criterion value consisting of a single numerical value (also referred to as the “determination criterion value” below). or a predetermined reference range defined by an upper and lower limit value (also referred to as the “determination criterion range” below). That is, the term “determination criterion” as used herein encompasses the terms “determination criterion value” and “determination criterion range.”

The method for comparing the indicator associated with a D-amino acid to a predetermined determination criterion is not particularly limited. When a determination criterion value is used as a determination criterion, the determination criterion value may be used as the upper limit of the indicator associated with the D-amino acid, and the determination may be made based on, e.g., whether the indicator associated with the D-amino acid in the subject is equal to or higher than the determination criterion value or whether it exceeds the determination criterion value. Alternatively, the determination criterion value may be used as the lower limit of the indicator associated with the D-amino acid, and the determination may be made based on, e.g., whether the indicator associated with the D-amino acid in the subject is equal to or lower than the determination criterion value or whether it falls below the determination criterion value. On the other hand, when a determination criterion range is used as a determination criterion, the determination may be made based on, e.g., whether the indicator associated with the D-amino acid in the subject stays within, exceeds, or falls below the judgment criterion range. Alternatively, the determination may be made based on, e.g., whether the indicator associated with the D-amino acid in the subject shifts over time from outside of the determination criterion range to within the determination criterion range, from within the determination criterion range to outside of the determination criterion range, persistently outside of the determination criterion range, or persistently within the determination criterion range, etc.

As the determination criterion for the method of the present invention, a single determination criterion value or a single determination criterion range may be used, or two or more determination criterion values or determination criterion ranges may be used in combination, or one or more determination criterion values and one or more determination criterion ranges may be used in combination.

Detection of Virus Infection and/or Virus Infection Disease

According to an embodiment, the method of the present invention includes making a determination on the detection of virus infection and/or virus infection disease in a subject using an indicator associated with a D-amino acid in the subject, and providing information about, e.g., the detection of virus infection and/or virus infection disease in the subject based on the results of the determination.

Specifically, a change in an indicator associated with a D-amino acid in a subject can be used to detect virus infection and/or virus infection disease in the subject and to provide information on the detection results. For example, when a decrease is observed in a laboratory value of the amount of a D-amino acid in blood, which is an indicator associated with a D-amino acid, of a subject suspected of influenza infection (e.g., a patient or a healthcare worker), it can be determined that the subject has become infected with virus and/or affected with virus infection disease, and the determination results can be provided as detection information of virus infection and/or virus infection disease. As another example, when a decreasing trend is observed in a laboratory value of the amount of a D-amino acid in blood of a subject suspected of influenza infection, it can be determined that the subject has become infected with virus and/or affected with virus infection disease, and the determination results can be provided as detection information of virus infection and/or virus infection disease.

In this case, the detection may be performed by comparing a laboratory value of an indicator associated with a D-amino acid in the subject with a criterion determined from the indicator associated with a D-amino acid in the past healthy state of the same subject or a criterion determined from the indicator associated with a D-amino acid in a patient or a population of patients known to have virus infection and/or virus infection disease. For example, a profile of D-amino acids and/or L-amino acids in the blood of a subject with virus infection and/or virus infection disease may differ from a profile of D-amino acids and/or L-amino acids in the blood of a subject without virus infection and/or virus infection disease. Based on this finding, the presence or absence of viral infection in a subject can be determined by comparing a laboratory value of an indicator associated with a D-amino acid in the subject with a predetermined criterion (e.g., a reference range or a clinical decision value) for the indicator associated with a D-amino acid. As a specific example, in the case where the amount of a D-amino acid in blood is used as an indicator for a D-amino acid, when a laboratory value of the amount of the D-amino acid in the blood of the subject is lower than a predetermined determination criterion, or when it shows a decreasing trend which is higher than a predetermined determination criterion, then it can be determined that the subject is positive for virus infection and/or virus infection disease.

When using, as an indicator for a D-amino acid, a value obtained by correcting the amount of the D-amino acid in blood with, e.g., another laboratory value, then the determination criterion or determination analysis method for detecting virus infection and/or virus infection disease based on a change (e.g., a decrease or an increase) in the amount of the D-amino acid in blood can be set or modified as appropriate according to the contents of the correction to be made to the amount of the D-amino acid in blood. For example, when a value corrected for the amount of a D-amino acid in blood as an inverse or multiplier is used as an indicator associated with the D-amino acid, then a determination criterion set based on a change (e.g., a decrease or an increase) in the amount of the D-amino acid in blood can be used as an inverse or logarithm.

There have been no reports of changes in indices associated with D-amino acids (e.g., a decrease or a decreasing trend in the amount of a D-amino acid in blood) in subjects having virus infection and/or virus infection disease, and it has not been known that there exists a relationship between the presence or absence of virus infection and/or virus infection disease and a change in an indicator associated with a D-amino acid (e.g., a decrease or a decreasing trend in the amount of a D-amino acid in blood). Therefore, the method of the present invention is extremely useful for the detection and diagnosis of virus infection and/or virus infection disease in clinical practice.

Stage Classification of Virus Infection and/or Virus Infection Disease

According to an embodiment, the method of the present invention includes making a determination on the stage classification of virus infection and/or virus infection disease in a subject using an indicator associated with a D-amino acid in the subject, and providing information about the stage classification of virus infection and/or virus infection disease in the subject based on the results of the determination.

As used herein, an exacerbation in the degree of symptoms of virus infection disease may be referred to as “aggravation” or, depending on the degree of exacerbation, as “becoming a moderate level” or “becoming a severe level” (in the case of, e.g., COVID-19, a condition requiring a ventilator or ECMO). In addition, as used herein, a reduction in the degree of symptoms may be referred to as “improvement,” “recovery,” or “remission,” and a state where the pathogen has been eliminated from the subject may be referred to as “healed.” Furthermore, as used herein, the course, result, and outlook of symptoms may be referred to as “outcomes.”

As used herein, the “stage classification” of a virus infection disease refers to classifying the stage of the target infection disease in a subject according to its degree of progression into, e.g., “incubation stage,” “acute (worsening) stage,” “recovery (improving) stage,” “healed,” or “sequelae stage.” In general, the stage of a disease is classified based on genetic (molecular-biological) testing, immunological testing, image diagnosis, or physical findings, and may be used as the basis for determining the severity of the disease, treatment options, and prognostic evaluation. The definitions and criteria for the stages of a disease may be corrected or standardized from the rules for the handling of various infection diseases established by, e.g., relevant academic groups or societies.

In this embodiment, the method of the present invention may include:
when the indicator associated with a D-amino acid in the subject has decreased, determining that the subject is in an aggravating state of viral infection,
when the indicator associated with a D-amino acid in the subject has increased, determining that the subject is in an improving state of viral infection, and/or
when the indicator associated with a D-amino acid in the subject has repeatedly decreased and increased and then converged to within a healthy reference range, determining that the subject is in a healing state of viral infection.

In this embodiment, the stage classification may be performed by comparing a laboratory value of an indicator associated with a D-amino acid in the subject with a determination criterion determined from an indicator associated with a D-amino acid in a patient who was affected with the virus infection disease and whose stage of the virus infection disease was classified.

Specifically, for example, the stage of a virus infection disease in a subject can be classified using a laboratory value of the amount of a D-amino acid in the blood of the subject as an indicator associated with the D-amino acid in the subject. Specifically, the profile of D-amino acids and L-amino acids in the blood of a subject with a virus infection disease may change according to the progression of the virus infection disease. Based on this finding, a laboratory value of an indicator associated with a D-amino acid in a subject may be compared to a determination criterion value (e.g., a criterion range or a clinical decision value) based on the amount of the D-amino acids in blood to thereby make a determination on the stage classification of virus infection disease, and provide information about corresponding treatments and outcomes. For example, an indicator associated with a D-amino acid in blood may be used, and a laboratory value in a subject may be compared to stages of the virus infection disease associated with determination criterion values to thereby make a determination on the stage classification of the virus infection disease in the subject, and the results of the determination may be provided as information about the stage classification of the virus infection disease in the subject.

Alternatively, for example, the stage of a virus infection disease in a subject can be classified based on a change in a laboratory value of the amount of a D-amino acid in the blood of the subject as an indicator associated with the D-amino acid in the subject. Specifically, a period during which a laboratory value of the amount of a D-amino acid in the blood of a subject shows a decrease and/or a decreasing trend can be classified as a condition or stage in which the subject is at an acute stage, aggravating, and/or becoming severe in the virus infection disease, or a condition or stage in which there is no improvement and/or recovery in the virus infection disease of the subject. In addition, a period during which a laboratory value of the amount of a D-amino acid in the blood of a subject shows an increase and/or an increasing trend can be classified as a condition or stage in which the subject is at a recovery stage or shows an improvement and/or recovery in the viral infection. According to an embodiment, when the amount of a D-amino acid in in the blood of a subject shows a transient decrease, increase, or repetition of decreases and increases and then converges to within a reference range of a healthy state, then the subject may be classified as being in remission, having a good outcome, or being in a healing stage of the virus infection disease. Since virus infection diseases progress from mild to moderate to severe as the patient continues to aggravate, the severity of the disease can also be classified based on the duration of the disease stage and the degree of changes in the amount of a D-amino acid in blood.

According to an embodiment, when a laboratory value of the amount of a D-amino acid in the blood of a subject, which is an indicator associated with a D-amino acid, remains decreasing, then the subject may be classified to be in the state where the virus infection disease is worsening and/or becoming severer, or to show no sign of improving and/or recovering from the viral infection disease. On the other hand, when a laboratory value of the amount of a D-amino acid in the blood of a subject once decreases and then begins to increase, then the subject may be classified to be in the state of improving or recovering from the viral infection disease.

According to an embodiment, a laboratory value of an indicator associated with a D-amino acid in a subject shows a transient decrease, increase, or repetition of decreases and increases and then converges to within a criterion range for the indicator associated with a D-amino acid of the same subject in a past healthy state or to within a criterion range for the indicator associated with a D-amino acid of a reference individual group in a healthy state, then the subject may be classified as being in remission, having a good outcome, or being in a healing stage of the virus infection disease.

A decrease or an increase in a laboratory value of an indicator associated with a D-amino acid in the subject may be determined using, as a determination criterion, a criterion value or criterion range for the indicator in the same subject in a past healthy state, or a criterion value or criterion range for the indicator in reference individual group in a healthy state, or a predetermined clinical decision value, and comparing a laboratory value of the indicator associated with a D-amino acid in the subject to the determination criterion. Alternatively, it may be determined by comparing laboratory values of the same subject at two or more different points in time.

When using, as an indicator for a D-amino acid, a value obtained by correcting the amount of the D-amino acid in blood with, e.g., another laboratory value, then the determination criterion or determination analysis method for classifying the stage of virus infection and/or virus infection disease based on a change (e.g., a decrease or an increase) in the amount of the D-amino acid in blood can be set or modified as appropriate according to the contents of the correction to be made to the amount of the D-amino acid in blood. For example, when a value corrected for the amount of a D-amino acid in blood as an inverse or multiplier is used as an indicator associated with the D-amino acid, then a determination criterion set based on a change (e.g., a decrease or an increase) in the amount of the D-amino acid in blood can be used as an inverse or logarithm.

There have been no reports of changes in indices associated with D-amino acids (e.g., a decrease or a decreasing trend in the amount of a D-amino acid in blood) in subjects having virus infection and/or virus infection disease, and it has not been known that there exists a relationship between the stage of virus infection and/or virus infection disease and a change in an indicator associated with a D-amino acid (e.g., a decrease or a decreasing trend in the amount of a D-amino acid in blood). Therefore, the method of the present invention is extremely useful for the detection and diagnosis of virus infection and/or virus infection disease in clinical practice.

Validation of Test Results and/or Diagnosis Results on Virus Infection and/or Virus Infection Disease

The method of the present invention can also provide other types of information based on the determination results of detection and/or stage classification of virus infection and/or virus infection disease. For example, according to an embodiment, the method of the present invention can also provide information about validation of test results and/or diagnosis results on virus infection and/or virus infection disease in the subject, based on the determination results of detection and/or stage classification of virus infection and/or virus infection disease mentioned above, which was carried out using laboratory values of the indicator associated with a D-amino acid in the subject.

PCR and antibody tests, which are conventionally used for viral infection tests, have a problem that false-negative results are often obtained due to the amounts of virus or proteins contained in a collected sample (e.g., nose and throat wipes or sputum), which is often below the measurement limit (Non-Patent Literature 6, 7). On the other hand, fluctuations in laboratory values for indicators associated with D-amino acids such as the amounts of D-amino acids in the blood of the subject differ in principle from those of such conventional test markers, since the ingestion, absorption, transport, distribution, metabolism (synthesis and degradation), excretion, and action of such D-amino acids may fluctuate due in part to the results of viral infection. Therefore, indicators associated with D-amino acids are highly useful in determining true/false positives. Specifically, if a subject is suspected of virus infection based on the environments and symptoms of the subject, but a negative result has been obtained in a given test, a false negative (Type II error) or true negative determination may be made using laboratory values for the indicator associated with a D-amino acid. In addition, since the phenomenon of a falling (decrease) in the amount of a D-amino acid in the blood of a subject is specific to viral infection, unlike direct detection of viruses or their fragments, the indicator for a D-amino acid can be used for distinguishing between D- and L-amino acids with respect to physiological changes in the host and can test the phenotype of a disease state. Thus, the indicator for a D-amino acid has special characteristics and is effective in detecting viral infection and/or determining the authenticity of test results.

The phrase “verification of the validity of a test or diagnosis result” as used herein means that when a subject is diagnosed by a clinical test that may involve false positive or false negative determination results, the validity of the diagnostic result is verified using another indicator with a different principle. Such clinical tests that may involve false positive or false negative determination results include: interviews and examinations of samples (e.g., pharyngeal secretions, sputum and respiratory tract secretions, urine, vaginal secretions, feces, blood, spinal fluid, etc.) collected from subjects (e.g., biochemical tests, serological tests, endocrine tests, microbiological tests, virological tests, culture tests, microscopic tests, genetic tests (e.g., PCR, hybridization, etc.), immunological tests (e.g., antigen and antibody detection methods), pathological tests, imaging tests (e.g., endoscopy, contrast medium test, ultrasonography, CT scan, MRI scan, etc.); and tests related to companion diagnostics to preliminarily examine the effects and side effects of specific drugs. As a specific example, when a subject has been determined to be true positive by antibody detection in a given paired serum, the subject can be determined to be a true positive if the virus infection and/or virus infection disease of the subject is determined to be positive, or to be false positive (Type I error) if the virus infection and/or virus infection disease of the subject is determined to be negative, based on the determination of an indicator associated with a D-amino acid, such as the amount of a D-amino acid in the blood of the subject. Conversely, when a subject has been determined to be negative, the subject can be determined to be false negative (Type II error) if the virus infection and/or virus infection disease of the subject is determined to be positive, or to be true negative if the virus infection and/or virus infection disease of the subject is determined to be negative, based on the determination of an indicator associated with a D-amino acid, such as the amount of a D-amino acid in the blood of the subject.

Validation of test and/or diagnostic results based on the results of detection and/or staging of viral infections and/or viral infections as described above is performed by comparison and analysis using a determination criterion (also referred to as a “criterion range” or “clinical decision value”) for the indicator. The determination criterion (criterion range or clinical decision value) that can be used in the present invention are set as subject-specific criterion value or clinical decision value by laboratory values of each individual subject in a healthy state, or as a generally 95% interval from the distribution center of laboratory values of a population of healthy individuals that satisfy certain criteria (reference individuals) or a population of subjects having infection or infection disease, or any other interval can be set according to the purpose. The determination criteria are generally used to make judgments about the diagnosis, prevention or treatment, as well as prognosis of a particular condition, and include diagnostic thresholds, treatment thresholds, and preventive medicine thresholds. These thresholds (cutoff values) can be set by case-control studies, empirical rules of clinical medicine, case-count studies, cohort studies, expert consensus, etc., using analytical data and results on predictive ability and judgment ability using ROC curves (Receiver Operating Characteristic curve), multivariate logistic regression models, Cox proportional hazards models, etc. For example, information on the results of verification of the validity of the test or diagnosis results of the subject can be provided by comparing various laboratory values described above with the results of the determination based on the indicator associated with a D-amino acid according to the method of the present invention. In addition, when an increase and a decrease in laboratory values of an indicator associated with a D-amino acid in the subject at any two or more points in time are defined as an increasing trend and a decreasing trend, respectively, a more detailed validation of the test and/or diagnostic results can be made by comparing and analyzing the laboratory values for the indicator associated with a D-amino acid in the subject at those two or more points in time.

Selection of Therapeutic Means for Virus Infection and/or Virus Infection Disease

According to an embodiment, the method of the present invention can provide information about selection of therapeutic means for virus infection disease in the subject, the determination results of detection and/or stage classification of virus infection and/or virus infection disease mentioned above, which has been made based on laboratory values of the indicator associated with a D-amino acid in the subject.

The phrase “selection of therapeutic means” as used herein refers to, in a subject diagnosed with a specific disease, selecting or prioritizing the most appropriate means from surgery, radiation therapy, chemotherapy, drug therapy, immunotherapy, diet therapy, exercise therapy, etc., or specifics from each technique (e.g., technique, administration method, etc.), and applying treatment to the subject so as to optimize the selected therapeutic means. Criteria and objectives for selection include curing disease, reducing or eliminating symptoms, arresting or slowing disease progression, preventing disease or symptoms, preventing underlying disease from worsening, avoiding or minimizing adverse effects, improving cost-effectiveness, and improving or maintaining quality of life (QoL).

According to an embodiment, before the method of the present invention is performed, subjects known to have virus infection disease are divided into those who respond to the therapeutic measures for the virus infection disease and/or those who do not respond to the measures, and criteria for the selection of therapeutic measures are set in advance based on measured values of an indicator associated with a D-amino acid in these subjects. And when the method of the present invention is performed, laboratory values of the indicator associated with a D-amino acid obtained from the new subject can be compared with the judgment criteria set based on said known subject, and the judgment results obtained can be provided as information on the selection of the therapeutic means for virus infection and/or virus infection disease in the subject.

According to one embodiment, the method of the present invention can provide information to aid in the selection of therapeutic measures for virus infection disease, using an indicator associated with a D-amino acid in the subject. In relation to the response or change in viral infection or viral infection to treatment, taking advantage of the different profiles of D-amino acids and L-amino acids in the blood of the subject, a laboratory value of the indicator associated with a D-amino acid is compared with a determination criterion (criterion range or clinical decision value) predetermined for prognosis, to thereby select the most appropriate means from, or assist in selecting it by prioritizing, e.g., treatment, including medication, oxygen therapy (nasal cannula, HFNC, CPAP, NPPV, etc., respiratory failure), surgical treatment, ventilatory management (mechanical ventilator, etc.), ECMO (extracorporeal membrane oxygenation), hemotherapy (dialysis, plasma exchange, apheresis, etc.), thrombosis control, kidney damage control, symptomatic treatment (antipyretic, antitussive, etc.) and dietary treatment. In addition, during the treatment phase, real-time monitoring of the indicator associated with a D-amino acid in a subject makes it possible to provide information to assist in selecting treatment measures at the next phase. Furthermore, in the event of a pandemic or bioterrorism, based on judgment using the indicator associated with a D-amino acid, it can be used for triage to determine and select the priority level of medical care and treatment for subjects and for risk assessment in each pandemic phase selected from the alert phase, pandemic phase, and transition phase. Antiviral drugs, which are used primarily for drug therapy, are drugs that exhibit the effects of inhibition of viral adsorption and entry into host cells, inhibition of intracellular shedding, nucleic acid synthesis, and protein synthesis, and inhibition of extracellular release of viruses, etc., while oxygen therapy, ventilator use, and ECMO are used for severely ill subjects.

According to one embodiment, based on prognostic predictions or determinations made using the indicator associated with a D-amino acid in a subject, the method of the present invention can provide information to select the most appropriate antiviral or other drugs as means for treatment of viral infection disease or to assist in prioritizing the treatment means. As a specific example, the indicator associated with a D-amino acid in a subject can be used for setting a determination criterion (criterion range or clinical decision value) for the effects, side effects, and adverse reactions of a given drug in advance, and for comparing it with a laboratory value of the subject to examine whether the drug should be administered. In addition, according to one embodiment, the indicator associated with a D-amino acid in a subject can be used for predicting and determining the effects, side effects, and adverse reactions after drug administration to the subject, and for providing information to assist in the continuation or discontinuation of administration, or in the determination of the dosage and timing of administration to the subject. Furthermore, the indicator associated with a D-amino acid in a subject can be used for providing information to assist in screening and/or determining the means to control the value of the indicator associated with a D-amino acid in the subject. Furthermore, an indicator associated with a D-amino acid in the subject can be used to provide information for screening means of modulating the value of the indicator associated with a D-amino acid in the subject.

Specific examples of drugs that can be used as a means for treating virus infection disease include, but are not limited to: therapeutic drugs for virus infection disease such as neuraminidase inhibitors (e.g., oseltamivir, zanamivir, peramivir, laninamivir), M2 protein inhibitors (e.g., amantadine), RNA polymerase inhibitors (e.g., favipiravir, molnupiravir), cap-dependent endonuclease inhibitors (e.g., baloxavir, marboxil), anti-herpesviruses (e.g., aciclovir, valaciclovir, famciclovir, amenamevir), anti-cytomegalovirus agents (e.g., ganciclovir, foscarnet, valganciclovir, etc.), anti-hepatitis B virus drugs (e.g., entecavir, tenofovir, lamivudine, adefovir, etc.), anti-hepatitis C virus drugs (e.g., sofosbuvir, ribavirin, ledipasvir, etc.), nucleoside-based reverse transcription inhibitors (e.g., tenofovir, emtricitabine, etc.), non-nucleoside-based reverse transcriptase inhibitors (e.g., rilpivirine, efavirenz, etc.), integrase inhibitors (e.g., elvitegravir, dolutegravir, etc.), protease inhibitors (e.g., darunavir, ritonavir, etc.), CCR5 inhibitors (e.g., maraviroc, etc.) antibiotics, Chinese herbal medicines (e.g., kakkon-to (kudzu decoction), shoseiryu-to (minor bluegreen dragon decoction), maou-to (ephedra decoction), etc.), acetaminophen, NSAIDs, antihistamines, immunosuppressants (e.g., steroids, baricitinib, etc.), neutralizing antibody drugs (e.g., remdesivir, casirivimab, imdevimab, sotrovimab, etc.), Janus kinase inhibitors (e.g., baricitinib), RNA synthase inhibitors (e.g., remdesivir), biologically active peptides (e.g., adrenomedullin, etc.), GM-CSF preparations (e.g., sargramostim, etc.), anticoagulants (e.g., heparin, etc.), antiparasitic agents (e.g., ivermectin, etc.), humanized anti-human IL-6 receptor monoclonal antibodies (e.g., tocilizumab, sarilumab, etc.), serine protease inhibitors (e.g., nafamostat, etc.), vaccines (e.g., DNA vaccines, RNA vaccines, attenuated vaccines, adenovirus vector vaccines, etc.), oral replacement fluids, infusions, blood transfusions, etc.

Others

The method of the present invention can also be used to provide various other types of information in addition to the above. For example, according to one embodiment, the method of the present invention can be used for providing information on screening for viral infection and/or virus infection disease and information on the diagnosis of their pathologies, based on the determination results regarding the detection and/or stage classification of viral infection and/or virus infection disease, which have been made using laboratory value of the indicator associated with a D-amino acid. In addition, according to one embodiment, the method of the present invention can be used for providing information on screening for efficacy, adverse effects, and adverse reactions in drug development, clinical trial decisions, alternative endpoints, etc., based on the determination results regarding the detection and/or stage classification of viral infection and/or virus infection disease, which have been made using laboratory value of the indicator associated with a D-amino acid. A plurality of types of information as explained above may be provided individually or simultaneously, depending on the purpose.

[System for Providing Information about Virus Infection and/or Virus Infection Disease (the System of the Present Invention)]

An embodiment of the present invention relates to a system for providing information about virus infection and/or virus infection disease in a subject by carrying out the method of the present invention (hereinafter also referred to as “the system of the present invention”).

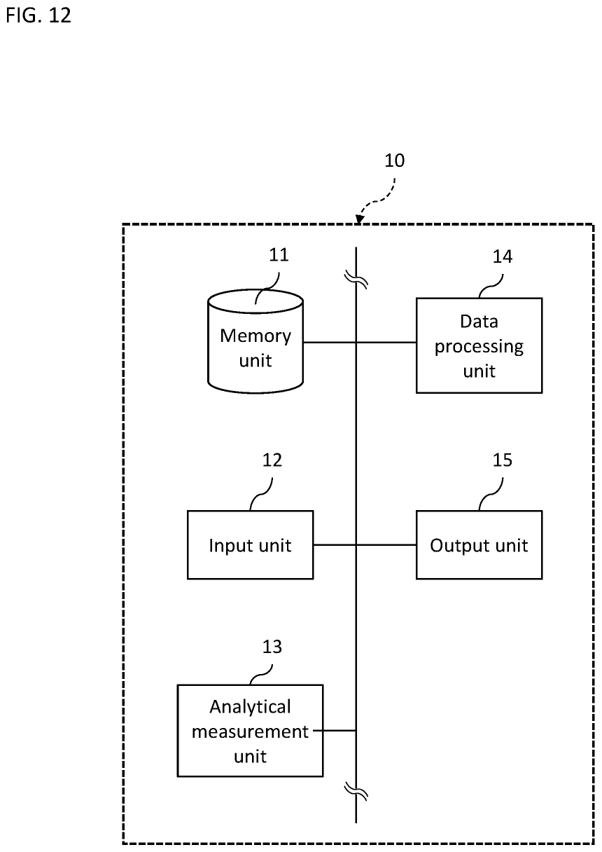

FIG. 12 is a block diagram schematically showing an example configuration of the system of the present invention. However, the configuration shown in FIG. 12 is presented for illustrative purpose only, and the configuration of the system of the present invention is in no way limited by this figure. The sample analysis system 10 shown in FIG. 12 includes a memory unit 11, an input unit 12, an analysis and measurement unit 13, a data processing unit 14, and an output unit 15. The memory unit 11 is configured to store various information including a determination criterion for virus infection and/or virus infection disease. The input unit 12 is configured to input various information including data from the subject. The analytical measurement unit 13 is configured to perform various analytical measurements, such as obtaining an indicator associated with a D-amino acid in the subject of the subject by analyzing and measuring data from the subject. The data processing unit 14 is configured to perform various arithmetic processes, such as performing a judgment on the viral infection and/or viral infection disease of the subject by processing the indicator associated with a D-amino acid of the subject based on the determination criterion. The output unit 15 is configured to output various information, such as information on the virus infection and/or virus infection disease.

Specifically, the memory unit 11 is composed of memory devices such as RAM, ROM, flash memory, etc., fixed disk devices such as hard disk drives, or portable storage devices such as flexible disks, optical disks, etc. The memory unit 11 is configured to store data and instructions input from the input unit 12, data measured by the analysis and measurement unit 13, results of arithmetic processing performed by the data processing unit 14, and various other information, such as computer programs and databases used in various processes of the information processing device realizing the sample analysis system 10. The computer program may be installed on a computer-readable recording medium, such as a CD-ROM or DVD-ROM, or via the Internet. The computer program is installed in the memory unit 11 using a known setup program or the like.

The input unit 12 is an interface with the outside of the sample analysis system 10, and also includes a keyboard, mouse, and other operating units. This allows the input unit 12 to input data measured in the analysis and measurement section 13, instructions for calculation processing to be performed in the data processing unit 14, and the like. The input unit 12 may also include, for example, an interface section that can input measured data, etc. via a network or a storage medium, in addition to the operation section, if the analytical measurement unit 13 is external.

The analytical measurement unit 13 is configured to obtain an indicator associated with a D-amino acid in the subject of the subject by analyzing and measuring information from the subject. For example, the analytical measurement unit 13 can be configured to measure at least the amount of a D-amino acid from a blood sample of the subject. Accordingly, the analytical measurement unit 13 can be configured to enable separation and measurement of D- and L-isomers of amino acids. Amino acids may be analyzed one at a time, or they may be analyzed collectively for some or all types of amino acids. Although not intended to be limitative, the analytical measurement unit 13 may be a chiral chromatography system, preferably a high-performance liquid chromatography system, equipped with a sample introduction section, an optical partitioning column, and a detection section. In terms of detecting only the amount of a specific amino acid, quantification may be performed by enzymatic or immunological methods. The analytical measurement unit 13 may be configured separately from the system for evaluating laboratory values, and the measured data and other data may be input via input unit 12 using a network or storage medium.

The data processing unit 14 can select information about the virus infection and/or virus infection disease in the subject by comparing the indicator associated with a D-amino acid measured by the analytical measurement unit 13 with the determination criterion stored in the memory unit. The indicator associated with a D-amino acid may be a formula or value corrected for the amount of a biological substance in the subject (e.g., the amount of a D-amino acid or a test index), or it may be a formula or value corrected for physiological variables such as age, sex, BMI, etc. The data processing unit 14 executes various arithmetic operations on the data measured by the analytical measurement unit 13 and stored in the memory unit 11 according to a program stored in the memory section. The arithmetic processing is performed by the CPU included in the data processing section. This CPU includes functional modules that control the analytical measurement 13, input unit 12, memory unit 11, and output unit 15, and can perform various types of control. Each of these parts may be composed of independent integrated circuits, microprocessors, software, etc.

The output unit 15 is configured to output information about the virus infection and/or virus infection disease in the subject, which is the result of the arithmetic processing in the data processing unit. The output unit 15 may be a display device such as an LCD display that directly displays the results of the arithmetic processing, a printer or other output means, or an interface section for output to an external storage device or over a network.

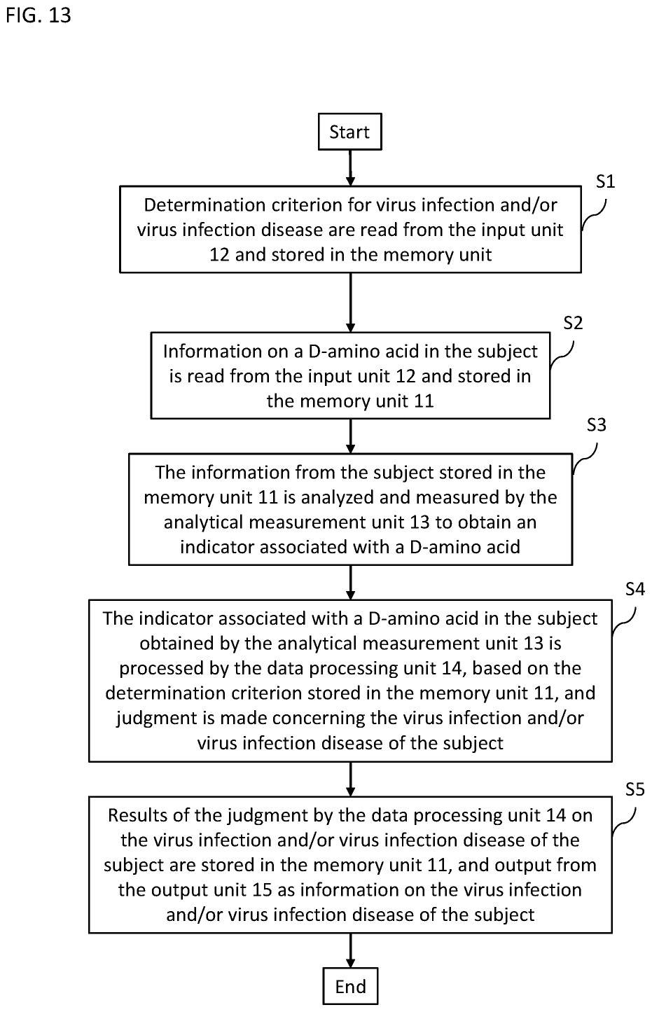

FIG. 13 is a flowchart schematically showing an example of processing by the system (the method of the invention). However, the process shown in FIG. 13 is presented for illustrative purpose only, and the processing by the system of the present invention is in no way limited by this figure. First, a determination criterion for virus infection and/or virus infection disease are read from the input unit 12 and stored in the memory unit 11 (step S1). Next, information on a D-amino acid in the subject is read from the input unit 12 and stored in the memory unit 11 (step S2). Then, the information from the subject stored in the memory unit 11 is analyzed and measured by the analytical measurement unit 13 to obtain an indicator associated with a D-amino acid in the subject (step S3). Next, the indicator associated with a D-amino acid in the subject obtained by the analytical measurement unit 13 is processed by the data processing unit 14, based on the determination criterion stored in the memory unit 11, and judgment is made concerning the virus infection and/or virus infection disease of the subject (step S4). The results of the judgment by the data processing unit 14 on the virus infection and/or virus infection disease of the subject are then stored in the memory unit 11, and output from the output unit 15 as information on the virus infection and/or virus infection disease of the subject (step S5).

### [Others]

The present invention has been described in detail in accordance with the specific embodiments above. However, the present invention is not limited to these embodiments, and a person skilled in the art can derive various other inventive concepts from the above description, all of which are within the technical scope of the present invention.

For example, a computer program for realizing the system of the invention and implementing the method of the invention using a general-purpose information processing device is provided (hereinafter also referred to as “the program of the present invention”). Specifically, the program of the present invention can be configured as a program including computer instructions that can be installed in a general-purpose information processing device and executed to cause the information processing device and external devices such as input/output interfaces and analysis devices connected thereto to function as, for example, the sample analysis system 10 shown in FIG. 12, which includes the memory unit 11, input unit 12, analytical measurement unit 13, data processing unit 14, and output unit 15. The program of the present invention can be realized with computer programming knowledge known to those skilled in the art. The program of the present invention and a recording medium such as a CD-ROM containing the program are also included in the technical scope of the present invention.

Since the present invention can be implemented by comparing laboratory values of the indicator associated with a D-amino acid in the subject with a predetermined determination criterion (criterion range or clinical decision value), it can be implemented by persons other than medical doctors, such as clinical examination, health checkup, and data processing companies, analysis systems, and analysis programs, without requiring judgment by a medical doctor, and thus does not fall under so-called medical activity, etc. Specifically, since it provides the determination result on detection and/or stage classification of virus infection and/or virus infection disease in a subject based on the indicator associated with a D-amino acid in the subject, it has extremely high technical utility as a preliminary or auxiliary method that does not replace medical practices such as diagnosis and treatment by a medical doctor, but improves the accuracy and efficiency of such diagnosis and treatment.

### EXAMPLES

The present invention will be described in more detail in the following examples. However, these examples are only shown for convenience of explanation, and the present invention is not limited to these examples in any sense. A person skilled in the art can easily make modifications and changes to the present invention based on the description herein, and all such modifications and changes shall be included in the technical scope of the present invention.

Symbols in the Examples have the following meanings.
PD-AA: D-amino acid concentration in plasma (nmol/mL, p,M)
PL-AA: L-amino acid concentration in plasma (nmol/mL, p,M)
PCre: Creatinine concentration in plasma
P % D: (PD-AA/PD-AA+PL-AA)×100 (%)

### Example 1: Influenza Model Mice Study

To induce severe infection in a short period of time in C57BL6 mice (SLC, Tokyo, Japan) raised in a specific pathogen control facility, 10 L of water containing 50 times the TCID50 (median tissue culture infectious dose, 50% infectious dose) of PR8 with H1N1 influenza virus strain (ATCC, Manassas, USA) was administered intranasally to anesthetized 4-week-old individuals (n=7) to induce infection. Blood was collected from each of the test individuals and control individuals (n=5) on a predetermined schedule after infection, and the plasma levels of chiral amino acids (D-amino acids and L-amino acids) were quantitatively analyzed by 2D-HPLC.

This experiment was approved by the Animal Committee of the National Institutes of Biomedical Innovation, Health and Nutrition (NIBIOHN) and was conducted in accordance with the guidelines of the Japanese Animal Protection and Control Law.

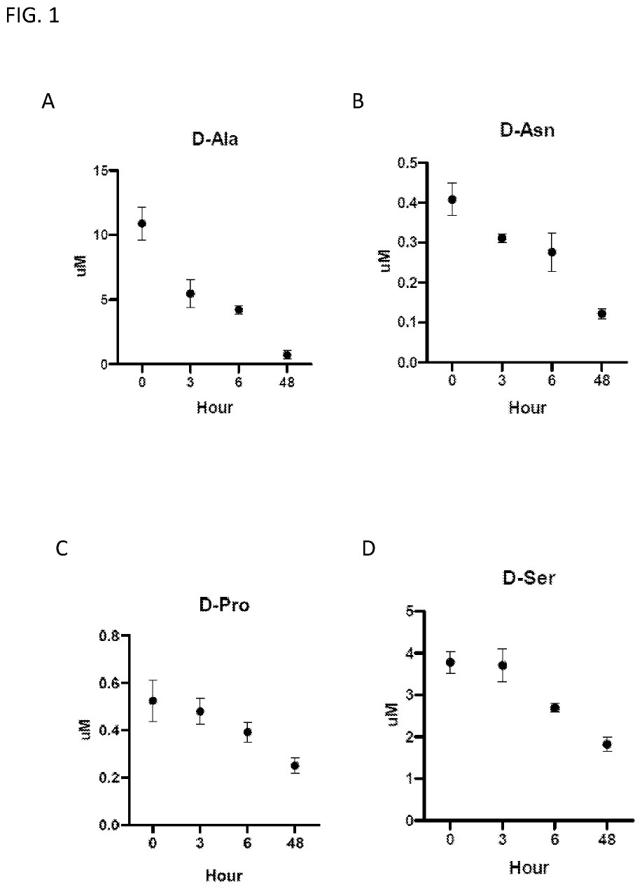

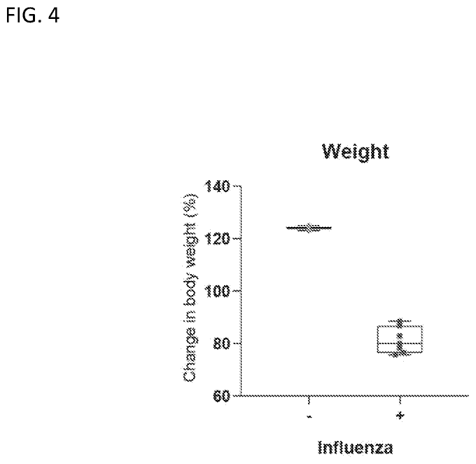

FIG. 1 shows the changes over time in the amounts of D-amino acids in the blood of influenza virus-infected mice in Example 1. FIG. 1A shows data for PD-Ala, FIG. 1B for PD-Asn, FIG. 1C for PD-Pro, and FIG. 1D for PD-Ser. As these data show, the amounts of these D-amino acids in the blood of influenza virus-infected mice all fell (decreased) over time after infection, making it possible to provide information on viral infection through changes in the blood D-amino acid levels. In addition, the amounts of the D-amino acids in the blood of the influenza virus-infected mice showed a continuous decline without turning to an increase, during which symptoms (weight loss, see FIG. 4) continued and the mice became severely ill and died. It is therefore deemed that the influenza virus-infected mice were in the acute (aggravation) stage throughout the duration of the post-infection study.

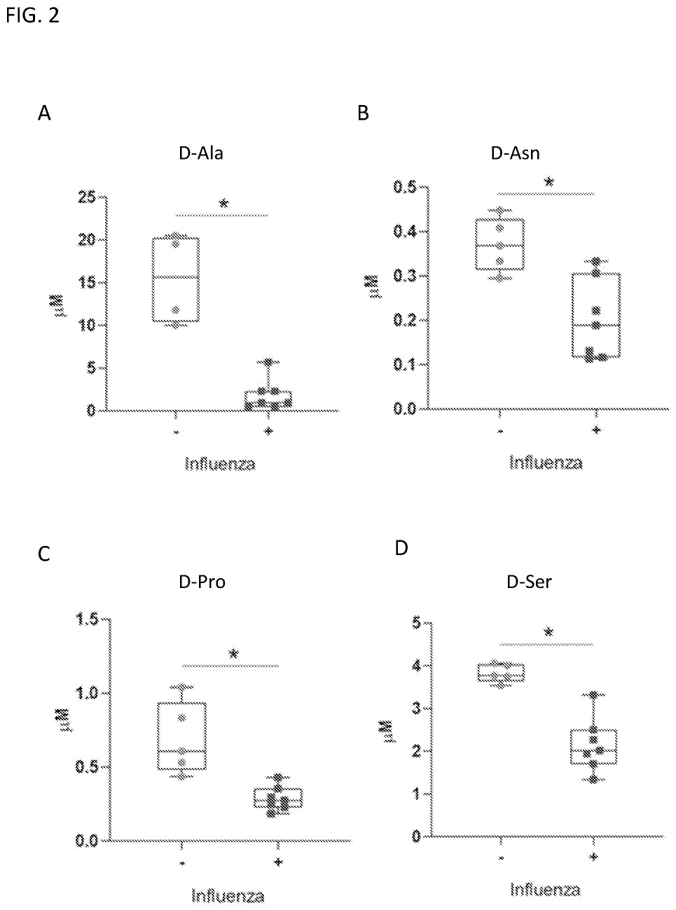

FIG. 2 indicates graphs showing the PD-AA levels in the influenza virus-infected and uninfected (control) mice groups in Example 1, with verification by t-test (*<0.05). FIG. 2A shows data for PD-Ala, FIG. 2B for PD-Asn, FIG. 2C for PD-Pro, and FIG. 2D for PD-Ser. These data show that PD-AAs of the influenza virus-infected mice significantly fell (decreased) compared to the control group in all cases, indicating that the viral infection can be determined based on the decrease in blood D-amino acid levels. A more accurate determination can be made by performing a panel test using multiple PD-AAs.

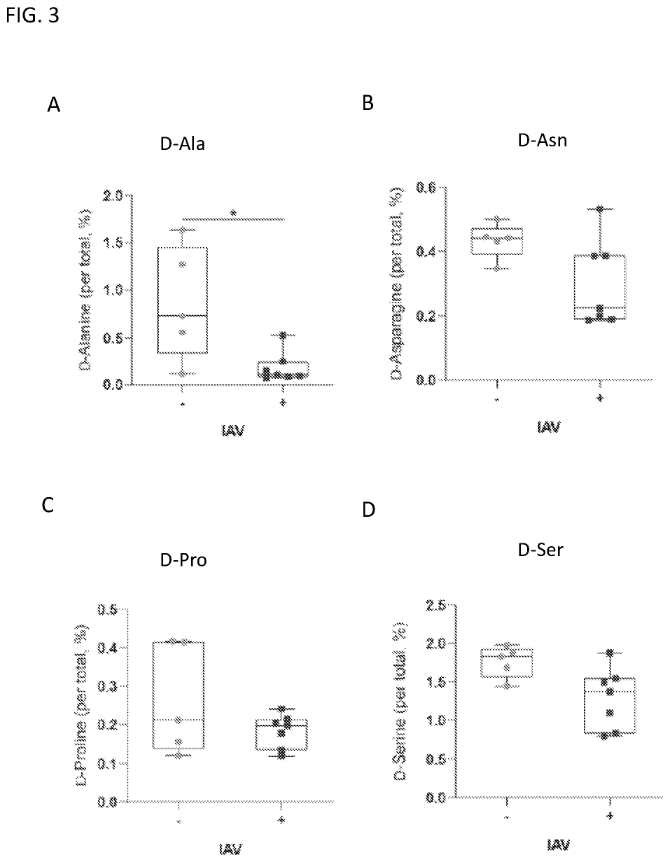

FIG. 3 shows indicates graphs showing the D-amino acid ratios in blood (P % D={PD−AA/(PD−AA+L−AA)}×100) of the influenza virus-infected and uninfected (control) mice groups in Example 1, with verification by t-test (*<0.05). FIG. 3A shows data for P % D-Ala, FIG. 3B for P % D-Asn, FIG. 3C for P % D-Pro, and FIG. 3D for P % D-Ser. These data show that P % D-Ala in the influenza virus-infected mice was significantly decreased compared to the control group, and P % D-Asn, P % D-Pro, and P % D-Ser each show a decreasing trend compared to the control group, indicating that viral infection can be determined based on the decreased blood D-amino acid ratios. A more accurate determination can be made by performing a panel test using multiple P % D-AAs. The determination accuracy can be further improved by conducting a panel test that combines the results of PD-AAs and P % D-AAs described above. The results of these determinations can also be used to validate the results of other tests for virus infection and/or virus infection disease, such as PCR tests.

FIG. 4 is a graph showing the body weights of the influenza virus-infected mice in Example 1 compared to the uninfected mice. These data show that the body weights, one of the symptoms of influenza virus infection, showed a clear reduction, indicating that the influenza model mice effectively serve as a model for severe influenza.

### Example 2: COVID-19 Clinical Study

An observational study was conducted as part of a Phase I and Phase II single-arm, multicenter, open-blind clinical study to evaluate the efficacy and safety of viral adsorption therapy in critically ill patients with novel coronavirus infection COVID-19 (CA1CH-COVID, Japanese clinical trial registration ID: 052200134).

Subjects were selected based on the following criteria. Selection criteria: patents with ages 18 years old or older, who showed SARS-CoV-2 positive blood in pre-registration tests, and severe cases of COVID-19 requiring either mechanical ventilation or ECMO (n=5, including one patient with renal failure). Exclusion criteria: patients with hemodynamic instability and inability to undergo hemodialysis. The primary outcome was discontinuation of ventilation or ECMO after recovery, and the secondary outcomes were (i) improvement in respiratory function as measured by the ratio of arterial partial pressure of oxygen to inspiratory partial pressure of oxygen (PaO2/FiO₂ ratio) and (ii) SARS-CoV-2 negative blood.

After screening by eligibility verification, including blood testing for SARS-CoV-2 on Day 0, patients received blood purification therapy using SARS-CoV-2 adsorption columns for 3 consecutive days. From Day 0 to Day 15, blood samples from eligible patients were analyzed quantitatively by HPLC for chiral amino acids (PD-AA and PL-AA) in plasma. A healthy population described in E. Clinical. Medicine, 2022, (Non-Patent Literature 3) and Sci. Rep. 2019, (Non-Patent Literature 8), which are among the present inventors' previously published articles, was used as a control.

This clinical study was approved by the Osaka University Central Ethics Review Board (#CRB5180007), written informed consent was obtained from all subjects, and the study was conducted in accordance with the Declaration of Helsinki and ethical guidelines for medical research involving human subjects.

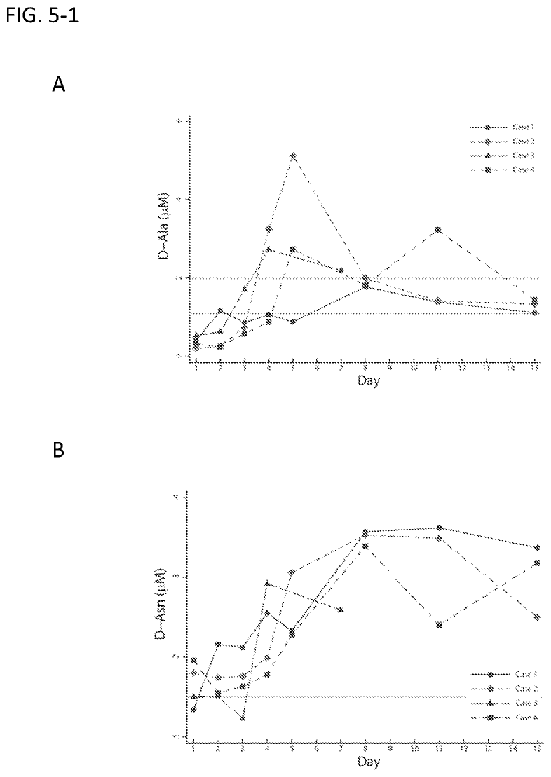

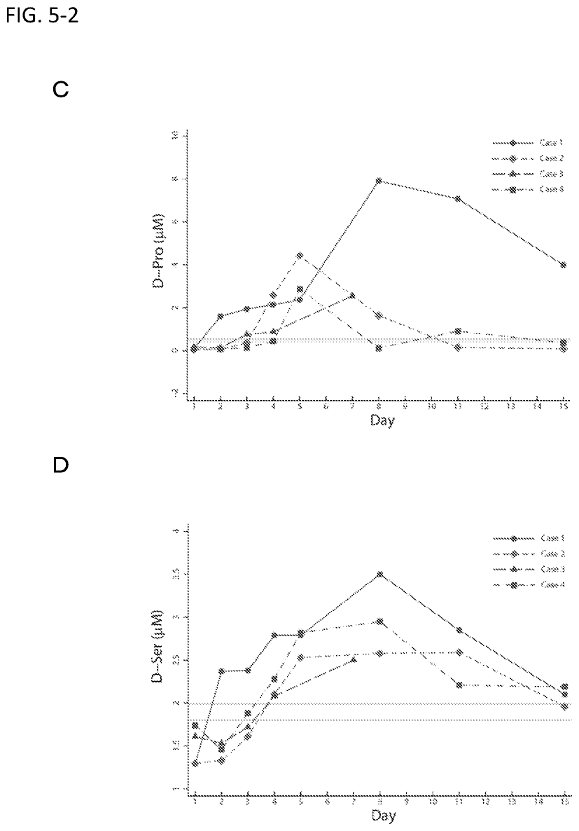

FIG. 5 indicates graphs showing the changes in the PD-AA levels over time during the course of treatment of COVID-19 patients in Example 2 (excluding patients with renal failure). FIG. 5A shows data for PD-Ala, FIG. 5B for PD-Asn, FIG. 5C for PD-Pro, and FIG. 5D for PD-Ser. The horizontal line indicates the criterion range (95% confidence interval) for healthy controls. As these data clearly show that the PD-AA levels of COVID-19 patients were lower than the criterion range or showed a decreasing trend, it is possible to determine whether the subject is affected by COVID-19 based on a decrease or a decreasing trend in blood D-amino acid levels. Although the patient group was severely ill requiring ECMO on Day 0, they recovered once the treatment (viral adsorption therapy) was initiated, and all patients eventually went into remission and were cured. The PD-AA levels showed a characteristic pattern of decreasing in the acute phase (aggravation stage or severe stage), followed by an increase in the recovery phase (improvement stage), and finally converging or approaching the criterion range, indicating that the amounts of D-amino acids in blood can be used to classify the stage of virus infection disease. The method can also assist in evaluating the effectiveness of therapies such as viral adsorption therapy and also in selecting effective therapies.

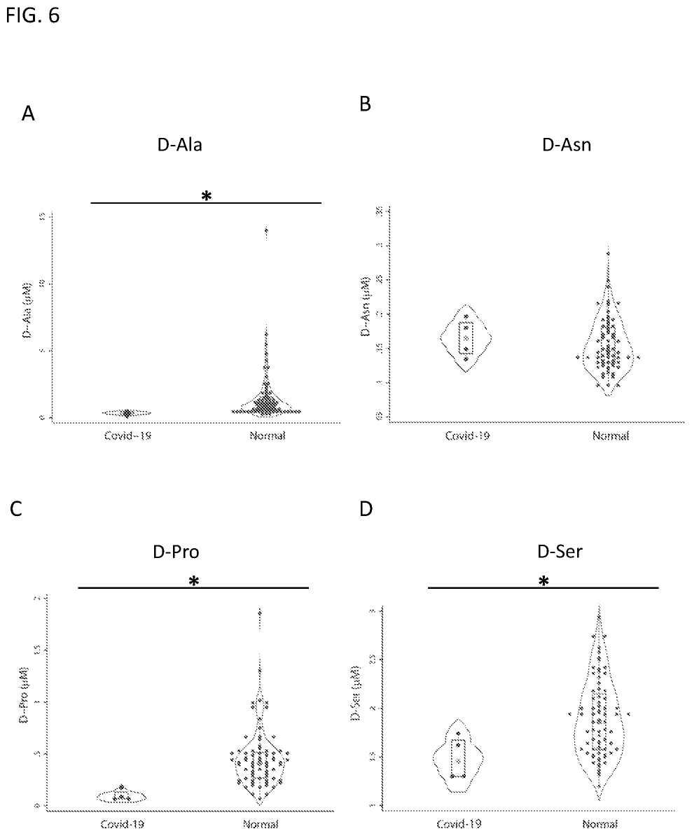

FIG. 6 indicates graphs showing the D-amino acid levels in the blood of the COVID-19 patient group and the healthy control group in Example 2, as verified by t-test. FIG. 6A shows data for PD-Ala, FIG. 6B for PD-Asn, FIG. 6C for PD-Pro, and FIG. 6D for PD-Ser. These data show that PD-Ala, PD-Pro, and PD-Ser in the COVID-19 patient group significantly fell (decreased) compared to the healthy control group, and PD-Asn showed a decreasing trend compared to the healthy control group, indicating that virus infection disease can be determined based on the decrease in blood D-amino acid levels. A more accurate determination can be made by performing a panel test using multiple PD-AAs.

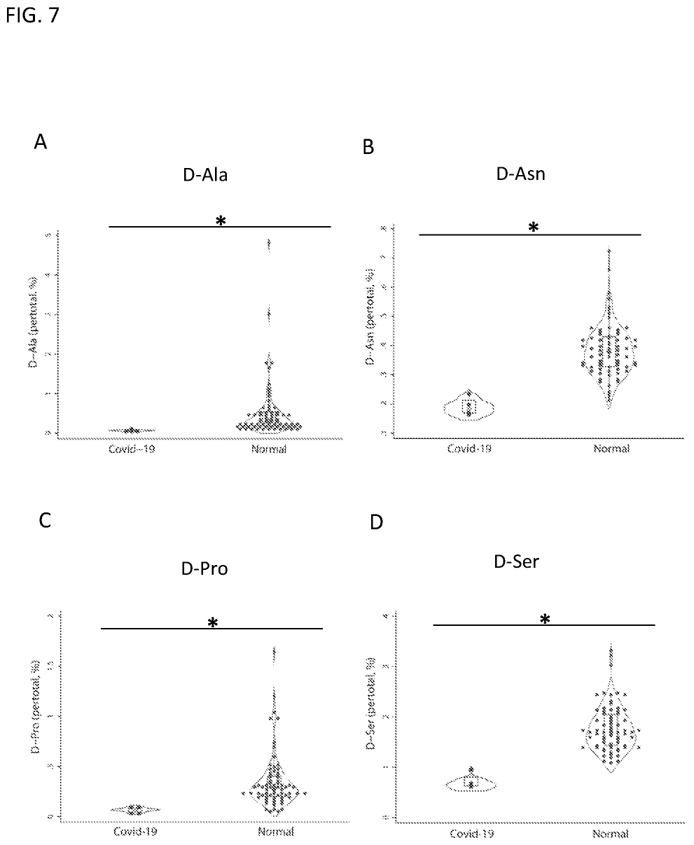

FIG. 7 indicates graphs showing the D-amino acid ratios in blood of the COVID-19 patient group and the healthy control group in Example 2 (% PD={PD−AA/(PD−AA+PL−AA)}×100) as verified by t-test (*<0.05). FIG. 7A shows the data for P % D-Ala, FIG. 7B for P % D-Asn, FIG. 7C for P % D-Pro, and FIG. 7D for P % D-Ser. These data clearly show that the P % D-AA of the COVID-19 patient group is significantly lower than that of the healthy control group in all cases, indicating that viral infection can be determined based on the decreased blood D-amino acid ratios. A more accurate determination can be made by performing a panel test using multiple P % D-AAs. The determination accuracy can be further improved by conducting a panel test that combines the results of PD-AAs and P % D-AAs described above. The results of these determinations can also be used to validate the results of other tests for virus infection and/or virus infection disease, such as PCR tests.

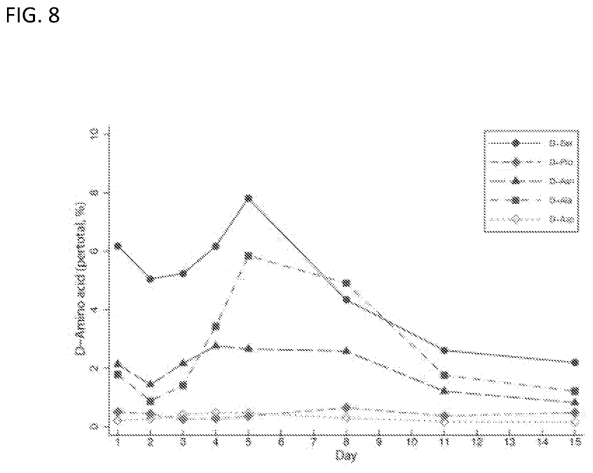

FIG. 8 indicates a graph showing the changes in PD-AA over time during the course of treatment of patients with end-stage renal failure, who were among the COVID-19 patient group but excluded from the above analysis in Example 2. These data show that PD-AA in end-stage renal failure patients affected by COVID-19 showed a characteristic pattern of variability similar to other COVID-19 patients, although the baseline was higher, as known from Patent Literature 5 and Non-Patent Literature 9. Therefore, the same evaluation and determination can be performed as in the examples shown in FIGS. 6 and 7 above by correcting for renal function markers (e.g., PCre, etc.).

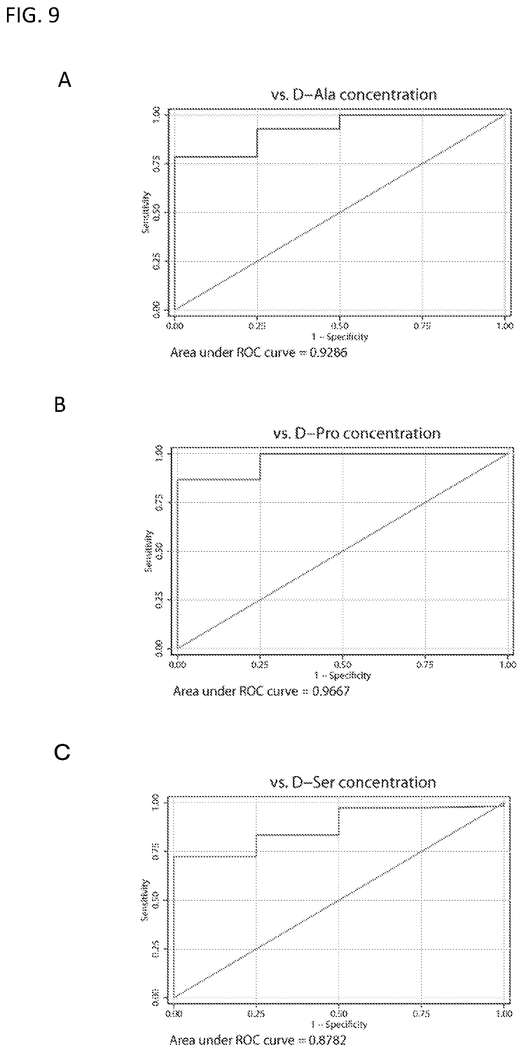

FIG. 9 shows the receiver operating characteristic (ROC) curves for the prediction (diagnosis) of COVID-19 with the PD-AA levels in the subjects as the variables in Example 2. FIG. 9A shows data for PD-Ala, FIG. 9B for PD-Pro, and FIG. 9C for PD-Ser. These data show that the area under the curve (AUC) when PD-Ala is used as a variable is 0.929, the AUC when PD-Pro is used as a variable is 0.967, and the AUC when PD-Ser is used as a variable is 0.878, indicating that all of these variables have high predictive (diagnostic) ability.

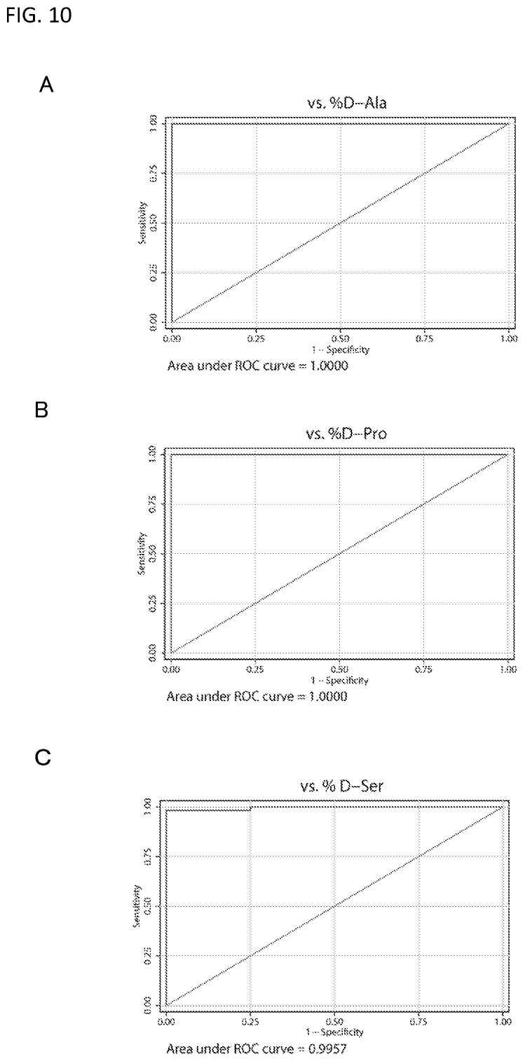

FIG. 10 shows the ROC curves for the prediction (diagnosis) of COVID-19 with the P % D-Ala values in the subjects as the variable in Example 2. FIG. 10A shows data for P % D-Ala, FIG. 10B for P % D-Pro, and FIG. 10C for P % D-Ser. These data show that the AUC is 1.000 when P % D-Ala is used as a variable, 1.000 when P % D-Pro is used as a variable, and 0.996 when P % D-Ser is used as a variable, indicating that all of them have extremely high predictive (diagnostic) ability.

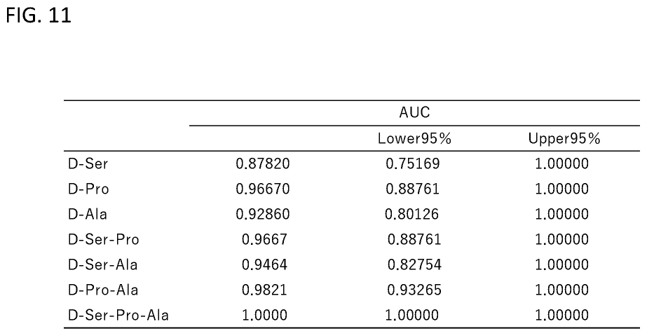

FIG. 11 is a table showing the AUC of the ROC curves for the prediction (diagnosis) of COVID-19 with multiple PD-AAs of the subjects in Example 2 as multiple variables. These data show that the AUC of PD-Ser*Pro is 0.967, PD-Ser*Ala is 0.946, and PD-Pro*Ala is 0.982 for two variables, and the AUC of PD-Ser*Pro*Ala is 1.000 for three variables, indicating that all of them have high predictive (diagnostic) ability.

### INDUSTRIAL APPLICABILITY

The present invention is highly useful in the field of diagnosis and treatment of virus infection and/or virus infection disease.

1. A method for providing information about virus infection and/or virus infection disease in a subject, comprising:
making a determination on the detection and/or stage classification of virus infection and/or virus infection disease in the subject using an indicator associated with a D-amino acid in the subject; and
providing information about virus infection and/or virus infection disease in the subject based on the results of the determination.

2. The method according to claim 1, wherein the indicator associated with a D-amino acid is a measurement value for the D-amino acid in blood or its correction value or correction formula.

3. The method according to claim 2, wherein the indicator associated with a D-amino acid is a value or formula obtained by correcting the amount of the D-amino acid by a parameter associated with a biological substance (e.g., an L-amino acid) in the subject.

4. The method according to claim 2, wherein the indicator associated with a D-amino acid is a value or formula obtained by correcting the amount of the D-amino acid by a parameter associated with a kidney function in the subject.

5. The method according to claim 1, wherein the D-amino acid is one or more D-amino acids selected from the group consisting of D-proline, D-serine, D-alanine, and D-asparagine.

6. The method according to claim 1, wherein the virus is a virus belonging to a family selected from Orthomyxoviridae, Coronaviridae, Paramyxoviridae, Rhabdoviridae, Arenaviridae, Bunyavirales, Filoviridae, Retroviridae, Togaviridae, Flaviviridae, Picornaviridae, Astroviridae, Caliciviridae, Reoviridae, Parvoviridae, Adenoviridae, Papillomaviridae, Polyomaviridae, Herpesviridae, Hepadnaviridae, and Poxviridae.

7. The method according to claim 1, wherein the determination on the detection of the virus infection and/or virus infection disease includes:
when the indicator associated with a D-amino acid in the subject has exhibited a decreasing trend, determining that the subject has become infected with virus and/or affected with virus infection disease.

8. The method according to claim 1, wherein the determination on the stage classification of the virus infection and/or virus infection disease is selected from:
the indicator associated with a D-amino acid in the subject has decreased, determining that the subject is in an aggravating state of viral infection,
the indicator associated with a D-amino acid in the subject has increased, determining that the subject is in an improving state of viral infection and/or
when the indicator associated with a D-amino acid in the subject has repeatedly decreased and increased and then converged to within a healthy reference range, determining that the subject is in a healing state of viral infection.

9. The method according to claim 1, wherein the determination on the detection and/or stage classification of the virus infection and/or virus infection disease is carried out by comparing the indicator associated with a D-amino acid in the subject with a determination criterion for virus infection and/or virus infection disease.

10. The method according to claim 1, wherein the information about virus infection and/or virus infection disease in a subject is information on an event selected from the group consisting of:
whether or not virus infection and/or virus infection disease in the subject is detected;
stage classification of virus infection disease in the subject;
validation of test results and/or diagnosis results on virus infection and/or virus infection disease in the subject; and
selection of therapeutic means for virus infection disease in the subject.

11. The method according to claim 10, wherein the therapeutic means for virus infection disease include means selected from antivirus drug, blood purification therapy, mechanical ventilation, and extracorporeal membrane oxygenation (ECMO).

12. A system for carrying out the method according to claim 1, comprising:
an input unit for inputting information from a subject;
an analytical measurement unit for analyzing and/or measuring the information from the subject inputted via the input unit to obtain an indicator associated with a D-amino acid in the subject;
a memory unit for storing a determination criterion for virus infection and/or virus infection disease;
a data processing unit for processing the indicator obtained by the analytical measurement unit for the subject based on the determination criterion stored by the memory unit to make a determination on virus infection and/or virus infection disease in the subject; and
an output unit for outputting the results of determination by the data processing unit as information about virus infection and/or virus infection disease in the subject.
